(19) United States
(12) Patent Application Publication
(10) Pub. No.: US 2026/0077085 A1
(43) Pub. Date: Mar. 19, 2026

(54) OPTICAL FLUID PROCESSING APPARATUS

(71) Applicant: KABUSHIKI KAISHA TOSHIBA, Kawasaki-shi (JP)

(72) Inventor: Hiroshi OHNO, Tokyo (JP)

(73) Assignee: KABUSHIKI KAISHA TOSHIBA, Kawasaki-shi (JP)

(21) Appl. No.: 19/312,784

(22) Filed: Aug. 28, 2025

(30) Foreign Application Priority Data
Sep. 18, 2024 (JP) 2024-161386

(51) Int. Cl. A61L 9/20

(52) U.S. Cl. CPC A61L 9/205; A61L 2209/12; A61L 2209/14

(57) ABSTRACT

According to an embodiment, an optical fluid processing apparatus includes a first reflector, a light source, a housing, and a heat sink. The first reflector includes a mirror surface. The light source is disposed to face the mirror surface of the first reflector. The housing includes an inlet and an outlet of a fluid. The first reflector and the light source are disposed inside the housing. The heat sink is thermally connected to the light source, and extended along a flow direction of the fluid flowing from the inlet to the outlet. A light beam from the light source includes a wavelength that is configured to inactivate a microorganism in the fluid.

### CROSS-REFERENCE TO RELATED APPLICATIONS

This application is based upon and claims the benefit of priority from Japanese Patent Application No. 2024-161386, filed Sep. 18, 2024, the entire contents of all of which are incorporated herein by reference.

### FIELD

Embodiments described herein relate generally to an optical fluid processing apparatus.

### BACKGROUND

In various fluids such as air and water, it is important to sanitize microorganisms such as filamentous fungi, bacteria, and viruses having pathogenicity or harmfulness, present therein. For example, an optical sanitization apparatus that illuminates a fluid with ultraviolet light using an ultraviolet ray (UV) light source to sanitize the fluid is known. Sanitization efficiency generally increases as intensity of the ultraviolet light applied to the fluid increases. However, the light from the light source immediately diverges, and the intensity decreases as a distance from the light source increases.

### BRIEF DESCRIPTION OF THE DRAWINGS

FIG. 1 is a schematic cross-sectional view illustrating an optical sterilization apparatus according to a first embodiment.

FIG. 2 is a schematic cross-sectional view illustrating a part of an optical fluid processing apparatus according to Modification 1 of the first embodiment.

FIG. 3 is a schematic cross-sectional view illustrating an optical fluid processing apparatus according to Modification 2 of the first embodiment.

FIG. 4 is a schematic cross-sectional view illustrating an optical fluid processing apparatus according to a second embodiment.

FIG. 5 is a schematic cross-sectional view illustrating an optical fluid processing apparatus according to a third embodiment.

### DETAILED DESCRIPTION

Hereinafter, each embodiment will be described with reference to the drawings. The drawings are schematic or conceptual, and a relationship between a thickness and a width of each portion, a ratio of sizes between portions, and the like are not necessarily the same as actual ones. In addition, even in the case of representing the same portion, dimensions and ratios may be represented differently from each other depending on the drawings. In the present specification and each drawing, elements similar to those described with respect to already described drawings are denoted by the same reference numerals, and detailed description thereof is appropriately omitted.

In the present specification, light is a type of electromagnetic wave, and includes X-rays, ultraviolet rays, visible light, infrared rays, microwaves, and the like. In the present embodiment, it is assumed that the light is ultraviolet light, and for example, a center wavelength is 280 nm. Note that the light is not limited thereto.

Here, “sanitization” means inactivation (also referred to as inactivity) of viruses, bacteria, and the like existing in the fluid, and terms such as “disinfection”, “sterilization”, “bacteria reduction”, or the like can also be used instead of “sanitization”. In embodiments, the term “sanitization” is used, but the term “sanitization” can be replaced with “disinfection”, “sterilization”, “bacteria reduction”, or the like.

An object of an embodiment is to provide an optical fluid processing apparatus that prevents a decrease in an efficiency of “sanitization”, “disinfection”, “sterilization”, “bacteria reduction”, or the like for light at a position away from a light source.

According to the embodiment, an optical fluid processing apparatus includes a first reflector, a light source, a housing, and a heat sink. The first reflector includes a mirror surface. The light source is disposed to face the mirror surface of the first reflector. The housing includes an inlet and an outlet of a fluid. The first reflector and the light source are disposed inside the housing. The heat sink is thermally connected to the light source, and extended along a flow direction of the fluid flowing from the inlet to the outlet. A light beam from the light source includes a wavelength that is configured to inactivate a microorganism in the fluid.

### First Embodiment

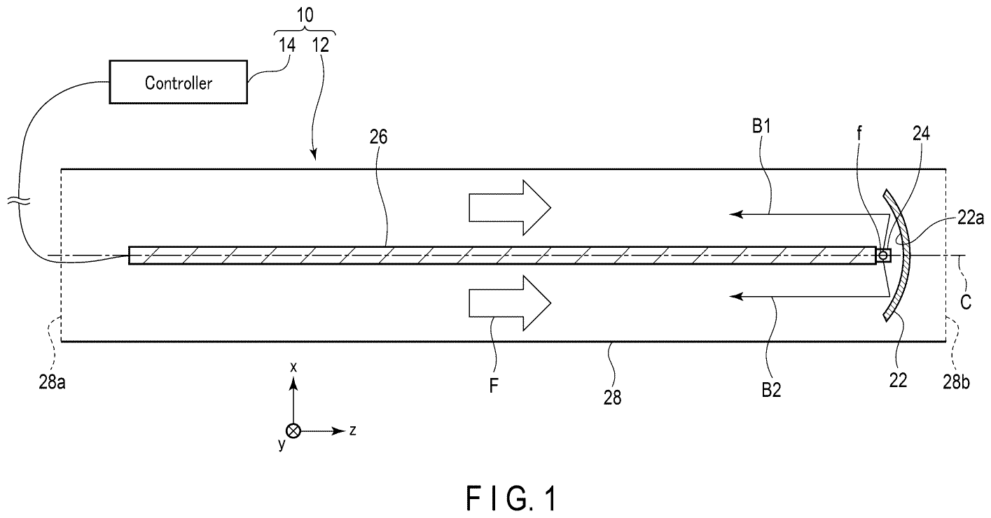

Hereinafter, an optical fluid processing apparatus (optical apparatus for sanitization, disinfection, sterilization, bacteria reduction, or the like) 10 according to a first embodiment will be described with reference to FIG. 1.

FIG. 1 is a schematic cross-sectional view of an optical fluid processing apparatus 10 according to the present embodiment. It is assumed that the cross-sectional view illustrated in FIG. 1 is on an x-z plane, and a z axis coincides with a central axis. It is assumed that an x axis is orthogonal to the z axis and is along the paper surface of FIG. 1.

The optical fluid processing apparatus 10 according to the present embodiment includes an apparatus body (optical fluid processing apparatus body) 12 and a controller 14.

In the present embodiment, the apparatus body 12 includes a first reflector 22 having a mirror surface 22a, a light source 24, a heat sink 26, and a housing 28 having an inlet (opening) 28a and an outlet (opening) 28b for fluid.

The first reflector 22 has a surface 22a that reflects light from the light source 24. The surface 22a of the first reflector 22 may be any surface as long as the surface reduces a divergence angle of the light from the light source 24. The first reflector 22 may be a concave mirror. For example, in FIG. 1, the first reflector 22 is schematically represented by one concave mirror, but may be configured by a combination of a plurality of lenses. Representative examples of the concave mirror 22 include a parabolic mirror, an elliptical mirror, an off-axis parabolic mirror, and an off-axis elliptical mirror. In the present embodiment, the first reflector 22 is a parabolic mirror. The parabolic mirror 22 has a focal point f. By disposing the light source 24 in a vicinity of the focal point f and making the parabolic mirror 22 and the light source 24 face each other, it is possible to convert the light from the light source 24 into parallel light by reflection by the parabolic mirror 22. The light from the light source 24 is reflected by the mirror surface 22a of the first reflector 22 to be close to parallel light. Therefore, the divergence angle of the light from the light source 24 is reduced.

Note that the mirror surface 22a of the first reflector 22 is formed in a concave shape in a +z-axis direction in FIG. 1. An axis passing through a center of the first reflector 22 is defined as a central axis C. It is assumed that the z axis coincides with the central axis C.

The reflector 22 may be an optical element having imaging performance. This is referred to as an imaging optical element. The imaging optical element 22 has a function to collect a light beam group emitted from one point into one conjugate point. This is also referred to as imaging or transfer to a conjugate point. A set surface of conjugate points at which a light beam group emitted from a sufficiently far point is transferred by the imaging optical element 22 is referred to as a focal plane of the imaging optical element 22. In addition, a line perpendicular to the focal plane and passing through the center of the imaging optical element 22 is defined as an optical axis C. At this time, a conjugate image point of an object point transferred by the light beam becomes the focal point. The reflector 22 may be an optical element having no imaging performance. This is referred to as a non-imaging optical element.

Reflectance of the mirror surface 22a of the reflector 22 is assumed to be, for example, equal to or higher than 90%. In the present embodiment, it is assumed that the reflector 22 is a parabolic mirror and has the reflectance equal to or higher than 90%.

A light emitting surface of the light source 24 is disposed so as to face the mirror surface 22a of the first reflector 22. It is assumed that the light source 24 is configured to emit light having a wavelength that inactivates microorganisms in a fluid toward the mirror surface 22a of the first reflector 22. Here, it is assumed that the light source 24 is configured to emit ultraviolet rays as the light having a wavelength that inactivates microorganisms in a fluid. Among the ultraviolet rays, a UV-C wavelength region is considered to have a high effect of inactivating microorganisms. Therefore, in the present embodiment, the light source 24 is an LED that emits UV-C. Note that the present embodiment is not limited thereto, and the light emitted from the light source 24 may be any light having a wavelength that inactivates microorganisms in the fluid. A light emitting area of the light emitting surface of the LED 24 is, for example, 5 mm×5 mm.

The light source 24 is configured to emit the light beam group having the divergence angle. For example, it is assumed that two light beams from the light source 24 include a first light beam B1 and a second light beam B2. At this time, an angle formed by these light beams B1 and B2 is defined as the divergence angle of the two light beams. The divergence angle of the total light beam group is the maximum of the divergence angles of the two light beams B1 and B2.

In a case where a point light source is disposed as the light source 24 at the focal point f of the parabolic mirror 22, the light beam group emitted from the point light source 24 is reflected by the parabolic mirror 22 and becomes a parallel light beam group. At this time, an axis parallel to the parallel light beam group and passing through the focal point f is the optical axis. Hereinafter, in the case where the light from the light source 24 is reflected by the reflector 22 to form the parallel light beam group, an axis parallel to the parallel light beam group and passing through the central axis C of the reflector 22 is defined as the optical axis. In the present embodiment, the central axis C of the reflector 22 and the optical axis coincide with each other.

The heat sink 26 is thermally connected to the light source 24. The heat sink 26 is connected to, for example, a region of the light source 24, the region being opposite to the light emitting surface. The heat sink 26 is extended along a flow direction F in which the fluid flows from the inlet 28a to the outlet 28b of the housing 28. In the present embodiment, the heat sink 26 is assumed to be, for example, a solid cylinder of aluminum. Note that the present embodiment is not limited thereto, and the heat sink 26 may be a triangular prism, a quadrangular prism, or a more polygonal prism. The heat sink 26 may have any shape made of a material having high thermal conductivity such as metal. Alternatively, the heat sink 26 may be a heat pipe provided with a refrigerant in a metal having a cavity. Note that, regarding dimensions of the heat sink 26, it is assumed that the maximum dimension along the central axis C is larger than the maximum dimension in a cross section orthogonal to the central axis C in a case where the maximum dimensions are compared. That is, regarding the dimensions of the heat sink 26, the maximum dimension along the flow direction F of the fluid is larger than the maximum dimension in the cross section orthogonal to the flow direction F of the fluid in a case where the maximum dimensions are compared, that is, the maximum dimension along the central axis C is larger. The heat sink 26 is assumed to be a rotationally symmetric body having a rotationally symmetric axis. The rotationally symmetric axis of the heat sink 26 is orthogonal to the light emitting surface of the light source 24.

In the present embodiment, the fluid is air. Note that the fluid in the present embodiment is not limited thereto, and any fluid may be used, such as water or rare gas. In a case where the apparatus body 12 according to the present embodiment is disposed in a space where the fluid is not retained, that is, a space where the fluid is moving, the fluid passes through the housing 28 of the present embodiment. It is assumed that the fluid enters the inlet 28a, flows along the flow direction F of the fluid indicated by reference sign F, and exits through the outlet 28b. Note that the inlet 28a and the outlet 28b of the housing 28 may be reversed. In this case, as a matter of course, the flow direction of the fluid indicated by reference sign F is also reversed to the direction illustrated in FIG. 1. In FIG. 1, the position indicated by reference numeral 28a is the inlet of the fluid, and the position indicated by reference numeral 28b is the outlet of the fluid. However, the inlet 28a may be disposed at a position close to the position where the reflector 22 and the light source 24 are disposed, and the outlet 28b may be disposed at a position far from the position where the reflector 22 and the light source 24 are disposed.

The housing 28 is formed of, for example, a metal material. The housing 28 is formed in a tubular shape provided with the inlet 28a for sucking the fluid and the outlet 28b. The housing 28 is preferably formed as a straight pipe. FIG. 1 illustrates an example in which the first reflector 22, the light source 24, and the heat sink 26 are disposed in the space between the inlet 28a and the outlet 28b of the housing 28. For example, the first reflector 22 may be disposed at a position away from the space between the inlet 28a and the outlet 28b of the housing 28. Further, the light source 24 together with the first reflector 22 may be disposed at a position away from the space between the inlet 28a and the outlet 28b of the housing 28.

It is assumed that the fluid flows in the housing 28 along the central axis C. The reflectance of an inner wall of the housing 28 may be any reflectance, but is assumed to be 60% here. That is, it is assumed that the reflectance of the inner wall of the housing 28 is lower than that of the mirror surface 22a of the reflector 22. In general, since the housing 28 has a larger surface area of its inner wall than the mirror surface 22a of the reflector 22, a cost increases in order to improve the reflectance of the housing 28. Further, the inner wall of the housing 28 often has a large curvature. Therefore, it is difficult to make the reflectance of the housing 28 higher than that of the mirror surface 22a of the reflector 22. Here, the shape of the inner wall of the housing 28 is assumed to be rotationally symmetric with respect to the central axis C. Note that the present embodiment is not limited thereto, and the housing 28 may have any shape for the inner wall. The shape of the inner wall of the housing 28 may be, for example, translationally symmetric in a direction perpendicular to the paper surface of FIG. 1.

In a case where the housing 28 is cylindrical and the reflector 22 is a parabolic mirror, an outer diameter of the reflector 22 is smaller than an inner diameter of the housing 28. Then, a gap through which the fluid passes is formed between an outer edge of the reflector 22 and an inner wall of the housing 28.

The controller 14 is electrically connected to the light source 24, for example. The controller 14 is used as a power source that supplies power to the light source 24 and switches ON/OFF of light emission of the light source 24. The controller 14 can adjust intensity of the light emitted from the light source 24. Wiring connecting the controller 14 and the light source 24 is provided, for example, along the central axis C. In this case, the wiring preferably passes through a hollow portion (cavity) of the heat sink 26.

Under the above configuration, an operation of the optical fluid processing apparatus 10 according to the present embodiment will be described.

As microorganisms in the fluid, there may be filamentous fungi, bacteria, viruses, and the like having pathogenicity or harmfulness. It is known that these microorganisms can be sanitized by irradiation with ultraviolet rays. Further, a sanitization effect of the fluid using the ultraviolet rays is larger as the intensity (luminance or illuminance) of the ultraviolet rays is larger, and the sanitization effect is larger as an irradiation time is longer. That is, a sanitization rate for the fluid is higher as the intensity of the ultraviolet rays is larger, and the sanitization rate for the fluid is higher as the irradiation time is longer.

First, consider a general optical fluid processing apparatus that reflects divergent light (ultraviolet light) from a light source (same as the light source 24 described in the present embodiment, for example) many times by an inner wall of a housing (same as the housing 28 described in the present embodiment, for example) without using the reflector 22 and the heat sink 26. For example, the light source 24 is disposed in the vicinity of the central axis C of the inlet 28a, and the light is emitted from the light emitting surface of the light source 24 along the flow direction F of the fluid. At this time, since the light emitted from the light source 24 is divergent light, many of light beams reach the inner wall of the housing 28 once and are reflected. Then, the light travels along the central axis C while further repeating the reflection on the inner wall of the housing 28, and reaches the vicinity of the outlet 28b of the housing 28. Due to such reflection, the intensity of the light beams is gradually reduced by the power of the reflectance. That is, in the vicinity of the outlet 28b of the housing 28, the intensity of the ultraviolet rays is the intensity obtained by raising the reflectance of the inner wall of the housing 28 to the power of the number of times of the reflection. As a result, the intensity of many of the light beams emitted from the light source 24 greatly attenuates before being emitted through the outlet 28b of the housing 28. Note that part of the light from the light source 24 is emitted through the outlet 28b of the housing 28 without being reflected by the housing 28.

Meanwhile, in a case where the general optical fluid processing apparatus includes the reflector 22 as in the apparatus body 12 of the optical fluid processing apparatus 10 according to the present embodiment, the light from the light source 24 is close to parallel light due to the reflection by the mirror surface 22a of the first reflector 22. Therefore, the apparatus body 12 of the optical fluid processing apparatus 10 according to the present embodiment can reduce the divergence angle of the divergent light from the light source 24. As a result, the number of times by which each of the light beams B1 and B2 is reflected by the inner wall of the housing 28 can be reduced as compared with the case where the reflector 22 is not provided. That is, for example, in a case where the intensity of light in the vicinity of the light source 24 is compared with, for example, the intensity of light at the inlet 28a of the fluid, it is possible to suppress attenuation of the intensity of the light beams B1 and B2 by using the reflector 22 as in the apparatus body 12 of the optical fluid processing apparatus 10 according to the present embodiment as compared with the case where the reflector 22 is not used. As described above, the sanitization effect of the fluid using the ultraviolet rays is larger as the intensity (luminance or illuminance) of the ultraviolet rays is larger. That is, there is an effect that the sanitization rate of the fluid flowing in the housing 28 can be increased even at a position away from the light source 24 by including the reflector 22 and causing the light from the light source 24 to be reflected by the mirror surface 22a of the reflector 22 to be close to parallel light.

The LED as the light source 24 generally has a heat resistant temperature of about 100 to 150° C. Therefore, to cause the light emitting surface of the light source 24 to continuously emit the light of appropriate intensity, a relatively large heat sink 26 is required. In the apparatus body 12 of the optical fluid processing apparatus 10 according to the present embodiment, the heat sink 26 thermally connected to the light source 24 is disposed in the vicinity of the light source 24. The heat sink 26 generally provides greater heat transfer to the fluid as its surface area in contact with the fluid increases. That is, the larger the surface area of the heat sink 26, the higher the heat dissipation performance. However, if the light source (LED) 24 is disposed at or in the vicinity of the focal point f of the reflector 22, and the heat sink thermally connected to the light source 24 is formed to be large in a radial direction orthogonal to the central axis C, for example, the light reflected by the reflector 22 and returned from the light source 24 is shielded by the heat sink although the heat dissipation performance is improved. Therefore, the sanitization efficiency for microorganisms in the fluid is attenuated.

In the present embodiment, the heat sink 26 is extended along the fluid flowing from the inlet 28a to the outlet 28b of the housing 28. Therefore, the surface of the heat sink 26 is always in contact with the fluid from a position connected to the light source 24 to a position far therefrom, and there is an effect that the heat dissipation performance is improved because the surface area of the heat sink 26 increases as the heat sink 26 is further extended.

In the apparatus body 12 according to the present embodiment, the first parallel light beam B1 and the second parallel light beam B2 formed by the reflector 22 propagate in the fluid in the housing 28 for a longer time as the housing 28 is extended and the heat sink 26 is further extended along the central axis C. That is, it is possible to lengthen an exposure time of light (ultraviolet light) to the microorganisms in the fluid in the housing 28. Therefore, it is possible to promote inactivation of the microorganisms in the fluid in the housing 28. That is, it is possible to improve the sanitization efficiency for the microorganisms in the fluid in the housing 28 by using the optical fluid processing apparatus 10 according to the present embodiment. By using the optical fluid processing apparatus 10 according to the present embodiment, there is an effect of preventing the light from reaching the housing 28 and being attenuated because the first light beam B1 and the second light beam B2 are parallel light beams parallel to the central axis C.

From the above description, it is possible to further extend the heat sink 26 along the central axis C by making the housing 28 longer, and the heat dissipation performance is improved. Further, at the same time, it is possible to lengthen the exposure time of light having appropriate intensity by which the attenuation due to the reflection on the inner wall of the housing 28 is suppressed with respect to the microorganisms in the fluid, and thus it is possible to improve the sanitization efficiency for the microorganisms in the fluid.

Meanwhile, if the heat sink is extended in the direction orthogonal to the central axis C, the light beam reflected by the reflector 22 is blocked by the heat sink although the heat dissipation performance is improved. Therefore, the sanitization efficiency for microorganisms in the fluid is attenuated. Further, if the heat sink is extended in the direction orthogonal to the central axis C, the flow of the fluid is easily hindered by the heat sink. Therefore, the heat dissipation performance is inferior to that of the heat sink 26 further extended along the central axis C as in the present embodiment. Meanwhile, as in the heat sink 26 according to the present embodiment, by further extending the heat sink 26 along the central axis C while maintaining the appropriate outer diameter of the heat sink 26, it is possible not to hinder the flow of the fluid and to promote a rectifying action of the fluid, and thus the heat dissipation performance is improved.

Regarding the dimensions of the heat sink 26, the maximum dimension along the central axis C is larger than the maximum dimension in the cross section orthogonal to the central axis C in a case where the maximum dimensions are compared. As a result, it is possible to prevent reflected light from the reflector 22 from being blocked and to make the heat dissipation performance equal to or higher than that in a case where the maximum dimension along the central axis C is smaller than the maximum dimension in the cross section orthogonal to the central axis C in a case where the heat sinks 26 having the same surface area are compared with each other. That is, regarding the dimensions of the heat sink 26, in the case where the maximum dimension along the flowing fluid and the maximum dimension in the cross section orthogonal to the maximum dimension are compared, the maximum dimension along the central axis C is larger. Therefore, it is possible to prevent the reflected light from the reflector 22 from being blocked and to make the heat dissipation performance equal to or higher.

Then, the heat sink 26 thermally connected to the light source 24 can continuously radiate heat generated by the light source 24. Therefore, the controller 14 can continue to emit the light of the appropriate intensity from the light emitting surface of the light source 24 with appropriate power. Therefore, by using the heat sink 26 thermally connected to the light source 24 and extended along the flow direction F of the fluid according to the present embodiment, it is possible to continuously emit the light with the appropriate intensity from the light emitting surface of the light source 24, and to continuously exhibit appropriate sanitization performance even at a position away from the light source 24.

Further, the optical fluid processing apparatus 10 according to the present embodiment makes the light from the light source 24 in the vicinity of the outlet 28b parallel by the reflector 22 and delivers the light to, for example, the inlet 28a while suppressing the attenuation in the housing 28. Therefore, by using the optical fluid processing apparatus 10 according to the present embodiment, it is possible to continuously exhibit the appropriate sanitization performance even at a position away from the light source 24.

Note that how to power the light source 24 is not limited to using the above-described controller 14. For example, a battery may be disposed along the heat sink 26.

Further, in the present embodiment, in the case where the housing 28 is cylindrical, the outer diameter of the reflector 22 is made smaller than the inner diameter of the housing 28, and the gap through which the fluid passes is formed between the outer edge of the reflector 22 and the inner wall of the housing 28. For example, if the mirror surface 22a of the reflector 22 has, for example, a plurality of through holes along the central axis C, the outer edge of the reflector 22 may be formed to be fitted to the inner wall of the housing 28. In this case, the gap through which the fluid passes may not be formed between the outer edge of the reflector 22 and the inner wall of the housing 28.

Further, a plurality of sets of the reflector 22, the light source 24, and the heat sink 26 may be disposed in one housing 28.

According to the present embodiment, it is possible to provide the optical fluid processing apparatus 10 that prevents a decrease in the sanitization efficiency for light at a position away from the light source 24.

### (Modification 1)

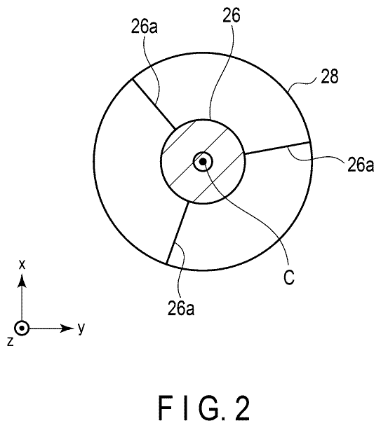

The optical fluid processing apparatus 10 according to Modification 1 of the first embodiment will be described with reference to FIG. 2.

FIG. 2 is a schematic cross-sectional view of the heat sink 26 and the housing 28 of the optical fluid processing apparatus 10 illustrated in FIG. 1 as cut along an xy cross section orthogonal to the z axis (central axis C) and viewing the inlet 28a side of the housing 28.

As illustrated in FIG. 2, the apparatus body 12 of the optical fluid processing apparatus 10 includes a fin 26a thermally connected to the heat sink 26 and the inner wall of the housing 28. The fin 26a is assumed to be made of a metal such as aluminum. As a result, heat is conducted from the heat sink 26 to the surface of the housing 28 via the fin 26a, and there is an effect that the heat dissipation performance is further enhanced. The housing 28 is preferably made of a material having high thermal conductivity such as aluminum. Note that the fin 26a may be integrally molded with the heat sink 26.

Further, if the fin 26a is formed as a connecting member that connects the heat sink 26 and the inner wall of the housing 28, an effect that the fin 26a can firmly fix the heat sink 26 to the inner wall of the housing 28 can also be expected.

Note that the fin 26a may not be thermally connected to the heat sink 26 and the inner wall of the housing 28. As long as the fin 26a extends from the heat sink 26 toward the inner wall of the housing 28, there is an effect that the heat is conducted from the heat sink 26 to the fin 26a and the heat dissipation performance can be enhanced as compared with a case where the fin 26a is not provided.

### (Modification 2)

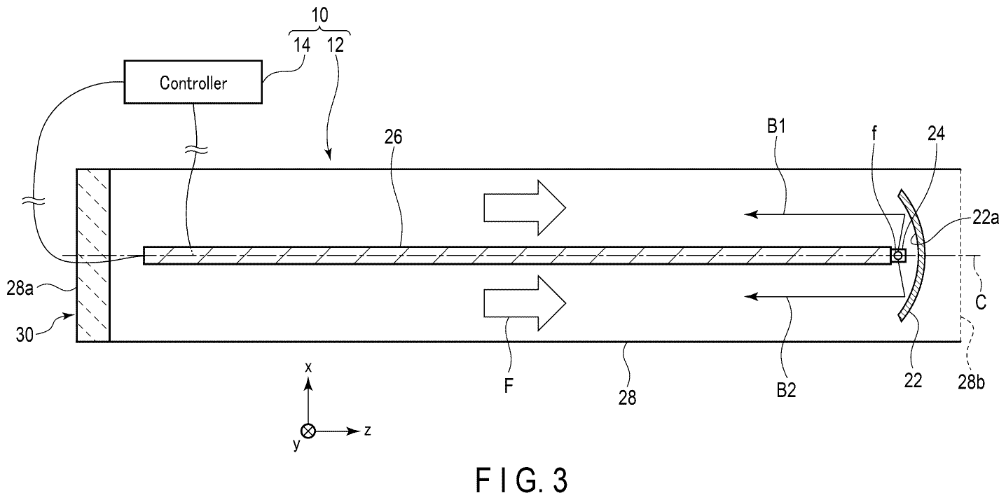

The optical fluid processing apparatus 10 according to Modification 2 of the first embodiment will be described with reference to FIG. 3.

As the light source 24 illustrated in FIG. 3, one or a plurality of LEDs that emits light including a UV-A wavelength region from the light emitting surface toward the mirror surface 22a of the first reflector 22 is used in addition to the UV-C wavelength region.

For example, a photocatalyst filter 30 in which a photocatalyst material is supported on a base formed of a porous material is disposed in, for example, the inlet 28a of the housing 28. As the base, for example, a ceramic porous body can be used. As the photocatalyst material, for example, titanium dioxide, zinc oxide, tungsten oxide, or the like can be used.

In this case, the light from the light source 24 is reflected by the mirror surface 22a of the first reflector 22 to be close to parallel light. Then, UV-C that suppresses the attenuation due to the reflection by the inner wall of housing 28 can be applied to the fluid in the space between the first reflector 22 and the photocatalyst filter 30. Similarly, UV-A that suppresses the attenuation due to the reflection by the inner wall of housing 28 can be applied to the fluid in the space between the first reflector 22 and the photocatalyst filter 30. Therefore, active oxygen and OH radicals are generated from the photocatalyst, and organic substances such as microorganisms in the fluid are decomposed into water and carbon dioxide. Note that the light emitted from light source 24 may include a wavelength that causes a chemical reaction such as an oxidation-reduction reaction with a substance in contact with the photocatalyst of the photocatalyst filter 30.

Therefore, even if the distance between the first reflector 22 and the photocatalyst filter 30 is appropriately long, it is possible to more reliably irradiate the photocatalyst filter 30 with UV-A, and to facilitate the reaction of decomposing the organic substances such as the microorganisms in the fluid passing through the photocatalyst filter 30 into water and carbon dioxide, by using UV-A that suppresses the attenuation due to the reflection on the inner wall of housing 28. In addition, it is possible to make a volume (space) for inactivating the microorganisms by UV-C large in the flow direction F along the central axis C. Therefore, it is possible to more efficiently perform both the photocatalytic reaction by the light irradiation with UV-A and the inactivation of the microorganisms and the like in the fluid by the light irradiation with UV-C. In other words, it is possible to achieve an appropriate sanitization rate with appropriate low ultraviolet intensity without considering the reflection of the ultraviolet rays by the inner wall of the housing 28 from the first reflector 22 to the inlet 28a. Therefore, it is possible to perform optical sanitization processing while suppressing power consumption by the light source 24.

According to the present modification, it is possible to provide the optical fluid processing apparatus 10 that prevents a decrease in the sanitization efficiency for light at a position away from the light source 24.

### Second Embodiment

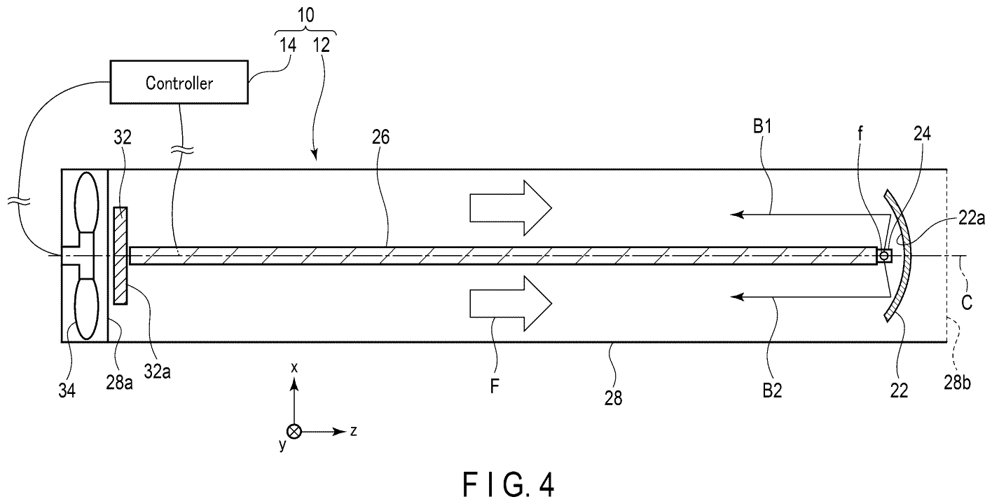

Hereinafter, an optical fluid processing apparatus 10 according to the present embodiment will be described with reference to FIG. 4.

FIG. 4 is a schematic cross-sectional view of an apparatus body 12 of the optical fluid processing apparatus 10 according to the present embodiment. In the present embodiment, the apparatus body 12 includes a first reflector 22, a light source 24, a heat sink 26, a housing 28, a second reflector 32, and a first fan 34.

The first reflector 22 according to the present embodiment is, for example, a parabolic mirror, and is formed in a similar manner to the first reflector 22 described in the first embodiment. The first reflector 22 has an optical axis C. The optical axis C coincides with a central axis of the first reflector 22.

It is assumed that the second reflector 32 includes, for example, a flat mirror surface 32a facing a mirror surface 22a of the first reflector 22. The flat mirror 32 may be, for example, a metal mirror made of aluminum.

Alternatively, the flat mirror 32 may be a dielectric multilayer film having heat conductivity, or a thin base having heat conductivity, to which reflection coating is applied. The flat mirror 32 is preferably orthogonal to the central axis C.

For example, an end of the heat sink 26, the end being farthest from the light source 24, is fixed to the flat mirror 32 of the second reflector 32. Note that the flat mirror 32 may have any shape, and is formed of, for example, a circular plate, an elliptical plate, or a polygonal plate.

The first fan 34 forcibly flows a fluid from an inlet 28a to an outlet 28b. The fan 34 may be any fan as long as the fan moves (flows) the fluid, but here it is assumed to be an axial fan having a turning axis.

Under the above configuration, an operation of the optical sterilization apparatus 10 according to the present embodiment will be described.

A controller 14 can drive the first fan 34 to forcibly move the fluid from the inlet 28a toward the outlet 28b of the housing 28. As a result, there is an effect that the fluid outside the housing 28 can be taken into the housing 28 and sanitized. In addition, there is an effect of adjusting a flow rate of the fluid by electrical control of rotation speed of the first fan 34 by the controller 14 and magnitude of a light amount by electrical control of the light source 24 to adjust sanitization efficiency of microorganisms outside the housing 28.

A parallel light beam group in which the light from the light source 24 is reflected by the mirror surface 22a of the first reflector 22 is reflected again by the flat mirror surface 32a of the second reflector 32, and reciprocates at least once along a same path along the central axis C. As a result, it is possible to increase an exposure time of microorganisms in the fluid taken into the housing 28 by the first fan 34 as compared with a case of one-way light. That is, there is an effect that the sanitization is improved.

In addition, the heat sink 26 can be forcibly cooled by the fluid sent from the first fan 34.

Therefore, according to the present embodiment, it is possible to provide the optical fluid processing apparatus 10 that prevents a decrease in the sanitization efficiency for light at a position away from the light source 24.

### Third Embodiment

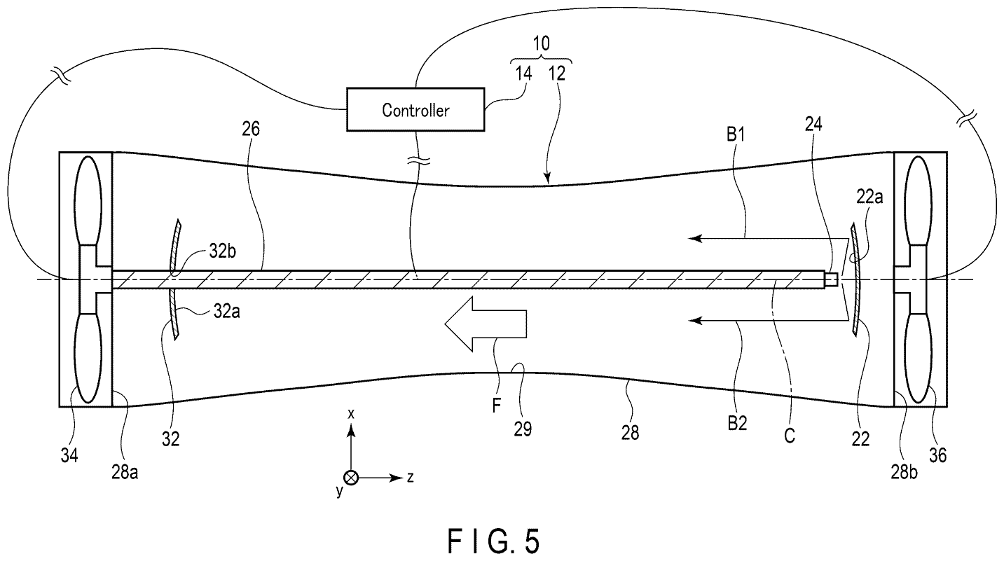

Hereinafter, an optical fluid processing apparatus 10 according to the present embodiment will be described with reference to FIG. 5.

FIG. 5 is a schematic cross-sectional view of the optical fluid processing apparatus 10 according to the present embodiment. The cross-sectional view illustrated in FIG. 5 is assumed to be on an xz plane.

An apparatus body 12 of an optical fluid processing apparatus 10 according to the present embodiment includes a first mirror (reflector) 22, a light source 24, a heat sink 26, a housing 28, a second mirror (reflector) 32, a first fan (blower) 34, and a second fan (blower) 36.

An axis passing through a center of the first mirror 22 is defined as an optical axis C. It is assumed that this cross-sectional view is on the x-z plane, and a z axis coincides with the optical axis C. In the present embodiment, it is assumed that an optical axis of the second mirror 32 and the optical axis of the first mirror 22 coincide with each other. Note that the optical axes in the present embodiment are not limited thereto, and the optical axes may be shifted from each other.

In the present embodiment, the second fan 36 sucks a fluid, and the first fan 34 discharges the fluid. That is, unlike the first fan 34 described in the second embodiment, the first fan 34 may discharge the fluid, and the second fan 36 may suck the fluid. The first fan 34 and the second fan 36 are, for example, axial fans, turning parts are axisymmetric, and respective symmetric axes and the optical axis C coincide with each other. Note that the present embodiment is not limited thereto, and the optical axis C and the respective symmetric axes of the first fan 34 and the second fan 36 may be shifted from each other. In addition, the fans 34 and 36 are not necessarily limited to axial fans, and may be any fans as long as they advect the fluid, such as centrifugal fans.

The heat sink 26 is disposed through the second mirror 32. That is, the second mirror 32 includes a through hole 32b in the center. The second mirror 32 may be, for example, a metal mirror made of aluminum. Alternatively, the second mirror 32 may be a dielectric multilayer film having heat conductivity, or a thin base having heat conductivity, to which reflection coating is applied.

The first mirror 22 and the second mirror 32 include reflective surfaces (mirror surfaces) 22a and 32a that reflect ultraviolet light. Both the reflective surfaces 22a and 32a have curved surfaces. The curved surface may have a radius of curvature larger than a distance between the two mirrors 22 and 32, for example. With such a radius of curvature, the light beams B1 and B2 can be reciprocated many times between the two mirrors 22 and 32. Note that, in the apparatus body 12 of the optical fluid processing apparatus 10 according to the present embodiment, at least two light beams (or beams) B1 and B2 are simultaneously reciprocated. As a result, it is possible to increase light beam illuminance of an xy cross section in the fluid as compared with a case of reciprocating one light beam (or beam).

Meanwhile, a method of obtaining concentration of an absorbent medium in a fluid by reciprocating one light beam (or beam) and obtaining an attenuation rate from an intensity ratio of the light beam before the reciprocation and after the reciprocation is known. In this case, if a plurality of light beams is simultaneously reciprocated, it is difficult to identify the light beam, and thus it is necessary to limit the number of light beams (or beams) to one.

The first mirror 22 is disposed in the vicinity of the symmetric axis of the turning part of the second fan 34. The second mirror 32 is disposed in the vicinity of the symmetric axis of the turning part of the first fan 34. Note that the disposition of the first fan 34 and the second fan 36 may be exchanged with each other.

An inner wall of the housing 28 has a structure 29 constricted near a center of the first fan 34 and the second fan 36. The inner wall of the housing 28 is assumed to be a rotationally symmetric body having a rotationally symmetric axis. The rotationally symmetric axis of the housing 28 is orthogonal to a light emitting surface of the light source 24.

Note that an outer diameter of the first mirror 22 and an outer diameter of the second mirror 32 are formed to be equal to or smaller than an inner diameter of the constricted structure 29 in the housing 28.

Under the above configuration, an operation of the optical fluid processing apparatus 10 according to the present embodiment will be described.

Consider a case where there is no second mirror 32. At this time, the light emitted from the light source 24, reflected by the first mirror 22, and traveling along the optical axis C cannot return to the first mirror 22 side again. On the other hand, by providing the curved second mirror 32 as in the present embodiment, the light reflected by the first mirror 22 and traveling along the optical axis C can be reflected by the reflective surface 32a of the second mirror 32 and travel to the reflective surface 22a on the first mirror 22 side again. In addition, the light is also typically sufficiently faster than a velocity of the fluid. That is, it can be considered that the fluid is substantially stopped while the light travels through the housing 28. Thereby, the light can pass through the same fluid at least twice, that is, an outward trip in which the light is reflected by the reflective surface 22a and travels toward the reflective surface 32a and a return trip in which the light is reflected by the reflective surface 32a and travels toward the reflective surface 22a. The reflective surfaces 22a and 32a according to the present embodiment can reciprocate the light beams B1 and B2 many times between the two mirrors 22 and 32. That is, it is possible to increase a stay time of the light in the same fluid. In other words, it is possible to lengthen an irradiation time with light. As a result, the optical fluid processing apparatus 10 according to the present embodiment has an effect of enhancing sanitization efficiency.

The first mirror 22 is disposed in the vicinity of the turning axis of the second fan 36, and the second mirror 32 is disposed in the vicinity of the turning axis of the first fan 34. In a case where an object is disposed in a region near the turning axis of the fan 34 or 36, the object is less likely to interfere with advection of the fluid by the fan 34 or 36 as a distance between the fan 34 or 36 and the object becomes shorter.

That is, it is possible to prevent an increase in pressure loss due to an object. Accordingly, for example, with respect to the second fan 36, by disposing the first mirror 22 in the region near the turning axis of the second fan 36, it is possible to prevent occurrence of the pressure loss. Similarly, with respect to the first fan 34, by disposing the second mirror 32 in the region near the turning axis of the first fan 34, there is an effect of preventing occurrence of the pressure loss.

The reflective surfaces 22a and 32a of the first mirror 22 and the second mirror 32 both have curvatures. These curvatures have a radius of curvature larger than the distance between the two mirrors 22 and 32. As a result, it is known that the light beam reaching the reflective surface 22a of the first mirror 22 from the light source 24 can reciprocate many times between the reflective surface 22a of the first mirror 22 and the reflective surface 32a of the second mirror 32. In the present embodiment, at least two light beams (first light beam B1 and second light beam B2) from the light source 24 are simultaneously reciprocated a plurality of times between the first mirror 22 and the second mirror 32. As a result, there is an effect that the illuminance of a fluid cross section by the light emitted from the light source 24 can be increased as compared with a case where one light beam is radiated one way and a case where one light beam is reciprocated once.

In the present embodiment, the fans 34 and 36 are axial fans. It is known that flow rate efficiency of the axial fans 34 and 36 is generally improved as diameters of the fans increase. In a case where the flow rate is constant, an amount of sanitization per unit time increases as the flow rate increases. Therefore, a synergistic effect of improving sanitization capability at the same time as improving the flow rate efficiency as the diameters of the axial fans 34 and 36 are increased can be expected.

The axial fans 34 and 36 are known to produce a swirl flow. The swirl flow increases the stay time of the fluid in the housing 28. As a result, the irradiation time with ultraviolet rays to the inside of the housing 28 becomes long, and there is an effect of improving the sanitization capability.

To efficiently generate the swirl flow, it is favorable to align the turning axes of the axial fans 34 and 36 and the rotationally symmetric axis of the heat sink 26 with each other. Since the heat sink 26 is rotationally symmetric, there is an effect that the swirl flow is more likely to occur than a case where the heat sink is not rotationally symmetric.

The inner wall of the housing 28 has the structure 29 constricted near the center of the first fan 34 and the second fan 36. Thus, the fluid passes inside the most constricted structure 29. This has an effect that the fluid can reliably pass through the light beam group by irradiating the vicinity of the inside of the constricted structure 29 or an inner wall portion of the constricted structure 29 with the light beams. That is, it is possible to reduce the fluid passing through the housing 28 without being irradiated with the light beams, and to reliably treat the fluid put in the housing 28 with the light beams.

For example, by disposing the first fan 34 and the second fan 36 at both ends of the housing 28 and using them for advection of the fluid, there is an effect of causing the fluid to flow at a large flow rate even in a case where the pressure loss of the fluid in the housing 28 is high.

The heat sink 26 is disposed in the vicinity of the first fan 34. As a result, it is possible to forcibly discharge, by the first fan 34, the fluid from the inside of the housing 28 to the outside of the housing 28 through an opening indicated by reference numeral 28a, so that heat dissipation of the heat sink 26 is promoted by the fluid advected from the inside of the housing 28 to the outside of the housing 28. As a result, it is possible to increase input power from the controller 14 to the light source 24, and there is an effect of increasing intensity of light and enhancing the sanitization capability of the fluid.

Therefore, according to the present embodiment, it is possible to provide the optical fluid processing apparatus 10 that prevents a decrease in the sanitization efficiency for light at a position away from the light source 24.

### (Modification)

A propeller surface of the turning part of the first fan 34 or the second fan 36 may be coated with reflection coating to reflect ultraviolet rays. Alternatively, a propeller of the turning part of the first fan 34 or the second fan 36 may be made of a material that reflects ultraviolet rays. As a result, by causing the light reaching the propeller of the first fan 34 or the second fan 36 to be reflected into the housing 28 and reciprocated in the fluid, it is possible to lengthen the stay time of the light in the fluid and enhance the sanitization capability.

According to the optical fluid processing apparatus 10 of at least one of the above-described embodiments, it is possible to prevent a decrease in an efficiency of “sanitization”, “disinfection”, “sterilization”, “bacteria reduction”, or the like for light at a position away from the light source 24.

While certain embodiments have been described, these embodiments have been presented by way of example only, and are not intended to limit the scope of the inventions. Indeed, the novel embodiments described herein may be embodied in a variety of other forms;

furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An optical fluid processing apparatus comprising:
a first reflector including a mirror surface;
a light source disposed to face the mirror surface of the first reflector;
a housing including an inlet and an outlet of a fluid, the first reflector and the light source being disposed inside the housing; and
a heat sink thermally connected to the light source, and extended along a flow direction of the fluid flowing from the inlet to the outlet, wherein
a light beam from the light source includes a wavelength that is configured to inactivate a microorganism in the fluid.

2. The optical fluid processing apparatus according to claim 1, wherein:
the light source is disposed at or near a focal point of the mirror surface of the first reflector.

3. The optical fluid processing apparatus according to claim 1, wherein:
a maximum dimension of the heat sink along the flow direction of the fluid is larger than a maximum dimension of the heat sink in a cross section orthogonal to the flow direction in a case where maximum dimensions are compared.

4. The optical fluid processing apparatus according to claim 1, comprising:
a fan disposed at one or both of the inlet and the outlet of the housing and configured to cause the fluid to flow along the flow direction of the fluid.

5. The optical fluid processing apparatus according to claim 1, wherein:
the light source is configured to emit an ultraviolet ray.

6. The optical fluid processing apparatus according to claim 1, wherein:
the heat sink has a rotationally symmetric axis, and the rotationally symmetric axis of the heat sink is orthogonal to a light emitting surface of the light source.

7. The optical fluid processing apparatus according to claim 6, comprising:
a fan disposed at one or both of the inlet and the outlet of the housing and configured to cause the fluid to flow along the flow direction of the fluid, wherein
the fan is an axial fan, and
a turning axis of the axial fan coincides with the rotationally symmetric axis of the heat sink.

8. The optical fluid processing apparatus according to claim 1, comprising:
a second reflector provided at a position spaced apart from the first reflector and facing the mirror surface of the first reflector, wherein
the light source is disposed between the first reflector and the second reflector.

9. The optical fluid processing apparatus according to claim 1, comprising:
a photocatalyst filter disposed at the inlet of the housing, wherein
the light source is configured to emit light having a wavelength that causes a chemical reaction with a substance in contact with a photocatalyst of the photocatalyst filter.
